(19) United States
(12) Patent Application Publication
(10) Pub. No.: US 2005/0256405 A1
(43) Pub. Date: Nov. 17, 2005

(54) Ultrasound-based procedure for uterine medical treatment

(76) Inventors: Inder Raj S. Makin, Loveland, OH (US); Yoav Avidor, Cincinnati, OH (US); Peter G. Barthe, Phoenix, AZ (US); Michael H. Slayton, Tempe, AZ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/847,209

(22) Filed: May 17, 2004

(51) Int. Cl. A61B 8/12

(52) U.S. Cl. 600/439; 600/437; 600/462; 601/2

(57) ABSTRACT

A first method for ultrasound uterine medical treatment includes obtaining an end effector having an ultrasound medical-treatment transducer assembly, identifying a blood vessel which supplies blood to a portion of the uterus, and medically treating the blood vessel with ultrasound from the transducer assembly to substantially seal the blood vessel to substantially stop the supply of blood from the blood vessel to the portion of the uterus. In one example, shrinkage of a uterine fibroid is accomplished through use of the end effector endoscopically inserted into the uterus. A second method for ultrasound uterine medical treatment includes endoscopically inserting the end effector into the uterus and medically treating the endometrium lining with ultrasound from the transducer assembly to ablate a desired thickness of at least a portion of the endometrium lining to substantially stop abnormal uterine bleeding from the endometrium lining.

### FIELD OF THE INVENTION

The present invention relates generally to ultrasound, and more particularly to a method for ultrasound uterine medical treatment.

### BACKGROUND OF THE INVENTION

Known ultrasound medical methods include using ultrasound imaging (at low power) of patients to identify patient tissue for medical treatment and include using ultrasound (at high power) to ablate identified patient tissue by heating the tissue. Known ultrasound imaging includes Doppler ultrasound imaging to detect blood flow, and a proposed known use of ultrasound includes using an ultrasound transducer outside the body to stop internal bleeding (by sealing ruptured blood vessels) of a patient brought to an emergency room of a hospital. It is known to inject an ultrasound contrast (echocontrast) agent, such as Optison™, into a blood vessel to better ultrasonically image the blood supply.

In one known ultrasound uterine medical treatment method, an ultrasound transducer is endoscopically inserted into the vagina but outside the uterus of a female patient and is used to ablate a portion of a uterine fibroid. Such treatment requires a relatively long treatment time because of the relatively large treated uterine-fibroid portion size and because the relatively large distance between the ultrasound transducer and the fibroid significantly attenuates the ultrasound focused at the uterine fibroid treatment site.

In another known ultrasound uterine medical treatment method, an ultrasound transducer outside the body of a female patient is used to ablate a portion of a uterine fibroid. In this method, only areas of the uterus not covered by the pelvic bone are capable of having their fibroids ultrasonically treated in this manner. Such treatment requires even a longer treatment time because of the relatively large treated uterine-fibroid portion size and because the relatively greater distance between the ultrasound transducer and the fibroid severely attenuates the ultrasound focused at the uterine fibroid treatment site.

In known non-ultrasound uterine medical treatment methods, blood flow in a uterine artery is blocked by embolization using small microspheres, by ligation, or by fulguration. The aim of such uterine artery blockage is to reduce the size of a uterine fibroid supplied by blood from the uterine artery and/or to reduce abnormal uterine bleeding from such uterine fibroid. Normal uterine tissue is essentially unaffected by these procedures as the uterus has abundant collateral blood supply via the uterine vasculature. However, having microspheres in an artery can pose potential medical problems, and ligating and fulgurating an artery are relatively invasive procedures.

In other known non-ultrasound uterine medical treatment methods, ablation of the endometrium lining is accomplished from within the uterus by a laser, by hot water, or by an expandable electrode. However, lasers, hot water, and electrodes have poor treatment depth control in ablating a desired thickness of endometrium lining, as can be appreciated by the artisan.

Known ultrasound medical systems and methods include deploying an end effector having an ultrasound transducer outside the body to break up kidney stones inside the body, endoscopically inserting an end effector having an ultrasound transducer in the colon to medically destroy prostate cancer, laparoscopically inserting an end effector having an ultrasound transducer in the abdominal cavity to medically destroy a cancerous liver tumor, intravenously inserting a catheter end effector having an ultrasound transducer into a vein in the arm and moving the catheter to the heart to medically destroy diseased heart tissue, and interstitially inserting a needle end effector having an ultrasound transducer needle into the tongue to medically destroy tissue to reduce tongue volume to reduce snoring. Rotatable ultrasonic end effectors are known which have an ultrasonic imaging transducer on one side and an ultrasonic treatment transducer on the opposite side and which have an ultrasonic treatment transducer of a short focal length on one side and an ultrasonic treatment transducer of a long focal length on the other side. A known ultrasonic end effector also includes a biopsy tool. Known methods for guiding an end effector within a patient include guiding the end effector from x-rays, from MRI images, and from ultrasound images obtained using the ultrasound treatment transducer.

Known non-ultrasound medical systems include endoscopic or laparoscopic clamp end effectors, wherein the clamp end effector is articulated and is steered by the user.

Still, scientists and engineers continue to seek improved methods for ultrasound uterine medical treatment.

### SUMMARY OF THE INVENTION

One method of the invention is for ultrasound uterine medical treatment of a female patient and includes steps a) through c). Step a) includes obtaining an end effector having an ultrasound medical-treatment transducer assembly. Step b) includes identifying a blood vessel which supplies blood to a portion of the uterus of the patient. Step c) includes medically treating the blood vessel with ultrasound from the transducer assembly to substantially seal the blood vessel to substantially stop the supply of blood from the blood vessel to the portion of the uterus. In one example, the ultrasound medical-treatment transducer assembly is an ultrasound imaging and medical-treatment transducer assembly. In one variation, the end effector is inserted (e.g., endoscopically or laparoscopically inserted) into the patient. In another variation, the end effector remains outside the patient. In one application, the portion of the uterus includes a uterine fibroid, and the blood vessel is a uterine artery or a branch thereof.

Another method of the invention is for ultrasound uterine medical treatment of a female patient and includes steps a) through e). Step a) includes obtaining an end effector having an ultrasound medical-treatment transducer assembly. Step b) includes transvaginal-endoscopically inserting the end effector into the patient. Step c) includes guiding the end effector within the patient into the uterine cavity. Step d) includes identifying the endometrium lining of the uterine cavity. Step e) includes medically treating the endometrium lining with ultrasound from the transducer assembly to ablate a desired thickness of at least a portion of the endometrium lining to substantially stop abnormal uterine bleeding from the endometrium lining.

Several benefits and advantages are obtained from one or more of the methods of the invention. In one example, ultrasound hemostasis (stoppage of blood flow) in a blood vessel supplying blood to a uterine fibroid is accomplished in a relatively non-invasive endoscopic or laparoscopic manner in a relatively short treatment time, because of the relatively small treated blood-vessel size and the relatively close distance between the ultrasound transducer and the blood vessel, which causes involution (shrinkage) of the uterine fibroid and stops any abnormal uterine bleeding from the uterine fibroid. In one extension, the uterine fibroid is also medically treated with ultrasound, in an endoscopic or laparoscopic manner, to ablate at least a part of the uterine fibroid. In another example, ultrasound ablation of at least a portion of the endometrium lining of the uterus is accomplished in a relatively non-invasive endoscopic manner and stops abnormal uterine bleeding from the endometrium lining of the uterus with excellent treatment depth control in ablating a desired thickness of endometrium lining because ultrasound can be focused to treat different depths within tissue.

The present invention has, without limitation, application in conventional endoscopic, laparoscopic, and open surgical instrumentation as well as application in robotic-assisted surgery.

### BRIEF DESCRIPTION OF THE FIGURES

FIG. 1 is a block diagram of a first method of the present invention for ultrasound uterine medical treatment;

FIG. 2 is a perspective view of an embodiment of an ultrasound medical treatment system having an endoscopic end effector used to perform one implementation of the first method of FIG. 1;

FIG. 3 is a view of a laparoscopic end effector used to perform another implementation of the first method of FIG. 1;

FIG. 4 is a block diagram of a second method of the present invention; and

FIG. 5 is a block diagram of a third method of the present invention.

### DETAILED DESCRIPTION OF THE INVENTION

Before explaining the present invention in detail, it should be noted that the invention is not limited in its application or use to the details of construction and arrangement of parts and/or steps illustrated in the accompanying drawings and description. The illustrative embodiments and methods of the invention may be implemented or incorporated in other embodiments, methods, variations and modifications, and may be practiced or carried out in various ways. Furthermore, unless otherwise indicated, the terms and expressions employed herein have been chosen for the purpose of describing the illustrative embodiments and methods of the present invention for the convenience of the reader and are not for the purpose of limiting the invention.

It is understood that any one or more of the following-described methods, implementations, applications, variations, modifications, etc. can be combined with any one or more of the other following-described methods, implementations, applications, variations, modifications, etc. For example, and without limitation, the third method illustration of ultrasound ablation of the endometrium lining of the uterus to stop abnormal uterine bleeding from the endometrium lining can be combined with the first method illustration of ultrasound hemostasis of a blood vessel supplying blood to a portion of the uterus containing a uterine fibroid to cause involution of the uterine fibroid, etc.

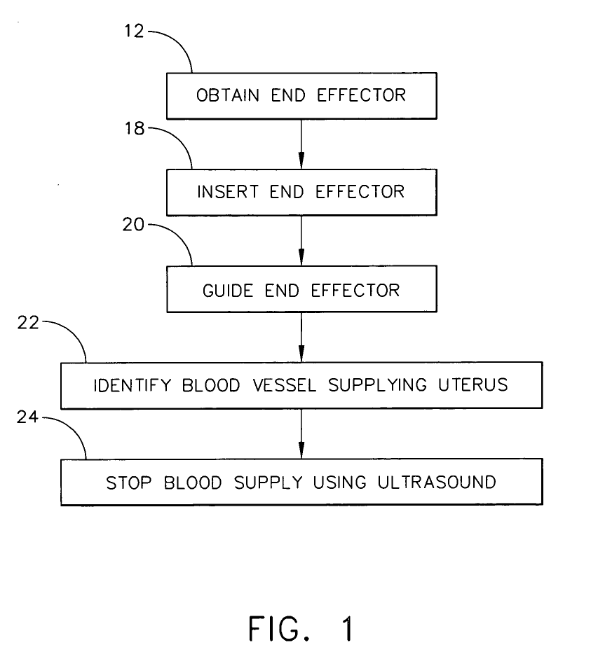

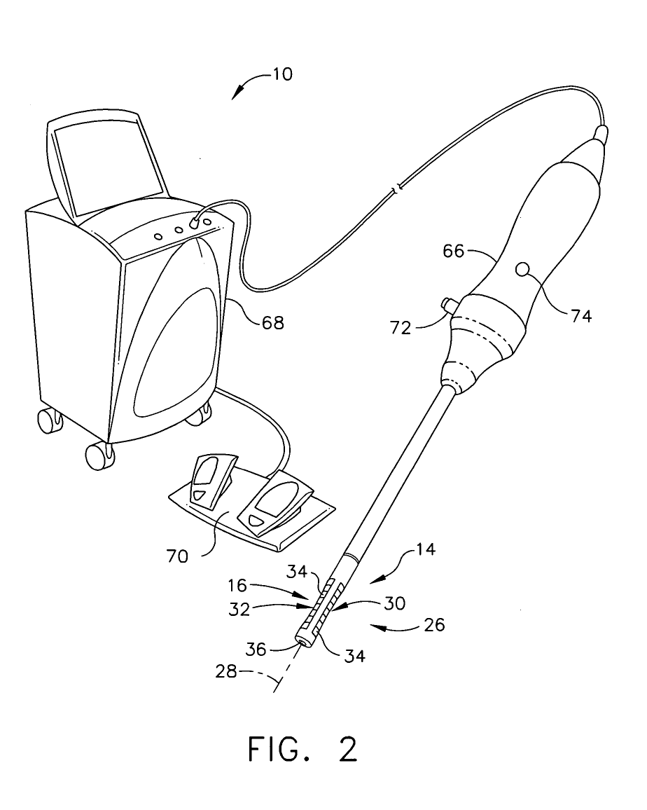

Referring now to the drawings, a first method of the invention is shown in block diagram form in FIG. 1 and is for ultrasound uterine medical treatment of a female patient. An embodiment of an ultrasound medical treatment system 10 which can be used to perform the first method is shown in FIG. 2. The first method includes steps a) through e). Step a) is labeled “Obtain End Effector” in block 12 of FIG. 1. Step a) includes obtaining an end effector 14 having an ultrasound medical-treatment transducer assembly 16. Step b) is labeled “Insert End Effector” in block 18 of FIG. 1. Step b) includes inserting the end effector 14 into the patient. Step c) is labeled “Guide End Effector” in block 20 of FIG. 1. Step c) includes guiding the end effector 14 within the patient to a region of patient tissue containing the uterus. Step d) is labeled “Identify Blood Vessel Supplying Uterus” in block 22 of FIG. 1. Step d) includes identifying a blood vessel in the region which supplies blood to a portion of the uterus. Step e) is labeled “Stop Blood Supply Using Ultrasound” in block 24 of FIG. 1. Step e) includes medically treating the blood vessel with ultrasound from the transducer assembly 16 to substantially seal the blood vessel to substantially stop the supply of blood from the blood vessel to the portion of the uterus.

An ultrasound medical-treatment transducer assembly 16 is an apparatus having at least one ultrasound transducer adapted at least for ultrasound medical treatment of a patient such as, but not limited to, a human patient. An ultrasound medical-treatment transducer includes either a single ultrasound medical-treatment transducer element or an array of ultrasound medical-treatment transducer elements, as is known to those skilled in the art. An ultrasound medical-treatment transducer may or may not also be adapted for ultrasound imaging of a patient.

In one implementation of the first method, step d) identifies the blood vessel from ultrasound imaging using the transducer assembly 16. In one variation, the blood vessel of step d) includes blood containing an ultrasound contrast (echocontrast) agent for improved ultrasonic imaging, as can be appreciated by the artisan. It is noted that Doppler ultrasound imaging alone, gray-scale ultrasound imaging alone, and a combination of Doppler and gray-scale ultrasound imaging are known ultrasound techniques to image blood flow in blood vessels.

In one application of the first method, the portion of the uterus includes a uterine fibroid, and the blood vessel is a uterine artery, or a branch thereof, which supplies blood to the uterine fibroid. In one variation, there is also included the step of medically treating the uterine fibroid with ultrasound from the transducer assembly 16 to ablate at least a part of the uterine fibroid. In one modification, there is also included the step of identifying the uterine fibroid at least in part from ultrasound imaging using the transducer assembly 16. In one option, the uterine fibroid in such identifying step includes blood containing an ultrasound contrast (echocontrast) agent for improved ultrasonic imaging, as can be appreciated by the artisan.

In the same or a different application of the first method, the portion of the uterus includes a source of abnormal uterine bleeding, and the blood vessel is a uterine artery, or a branch thereof, which supplies blood to the source of abnormal uterine bleeding. In one variation, the source is a uterine fibroid. In another variation, the source is not a uterine fibroid.

In one implementation, the end effector 14 is an endoscopic end effector 26 as seen in FIG. 2. In one variation, step c) guides the endoscopic end effector 26 into the uterine cavity. In one construction, the endoscopic end effector 26 has a longitudinal axis 28, has at least two transversely-outwardly-facing ultrasound transducers 30 and 32 each having at least one ultrasound transducer element 34, and has a longitudinally-facing ultrasonic tip transducer 36.

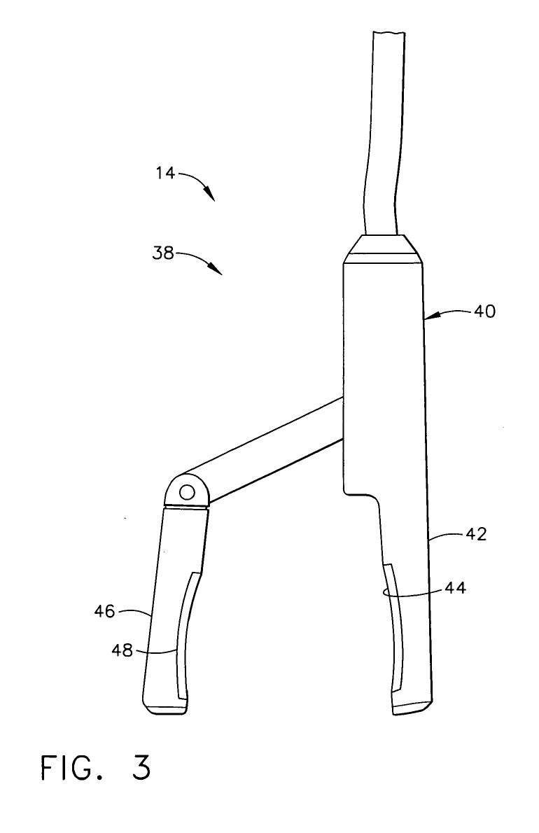

In another implementation, the end effector 14 is a laparoscopic end effector 38 as seen in FIG. 3. In one variation, the laparoscopic end effector 38 comprises a tissue-retaining device 40 including a first tissue-retaining member 42 having an ultrasound medical-treatment transducer 44 and including a second tissue-retaining member 46. In this variation, step e) includes retaining the blood vessel between the first and second tissue-retaining members 42 and 46. The first and second tissue-retaining members 42 and 46 are operatively connected together to retain the blood vessel between the first and second tissue-retaining members 42 and 46 and to release the blood vessel so retained. In one modification, the second tissue-retaining member 46 includes an ultrasound reflector 48. Choices of ultrasound reflecting materials/constructions include, without limitation, acoustically-rigid materials such as stainless steel (which reflects about 100%) and aluminum (which reflects about 80%), acoustically-compliant materials such as Corporene, and air-backed reflector elements. In one embodiment, the ultrasound reflector 48 is oriented to reflect the received ultrasound energy away from the transducer 44 when the blood vessel is retained by the tissue-retaining device 40. In a different modification, the second tissue-retaining member 46 does not include an ultrasound reflector. In one embodiment, the tissue-retaining device 40 is a clamp, and in another embodiment, the tissue-retaining device 40 is not a clamp. Mechanisms, not shown, for remotely moving two (or more) members toward and away from each other are within the ordinary level of skill of the artisan and include, without limitation, the use of pivotal member attachments and the use of cables or motors. In one variation, the first and second tissue-retaining members 42 and 46 are controllably orientatable relative to each other. In another variation, the first and second tissue-retaining members 42 and 46 remain parallel to each other during opening and closing.

In an additional implementation, not shown, the end effector 14 is a catheter end effector (such as, but not limited to, an intravascular catheter end effector). In one variation, step c) guides the catheter end effector inside the blood vessel. In one modification, the catheter end effector is first endoscopically inserted into the vagina and then is intravascularly inserted into the blood vessel.

In a further implementation of the first method, not shown, the end effector 14 is an open-surgery end effector. In a different implementation, the end effector 14 is a needle end effector. In one extension of the first method, the end effector 14 is used for ultrasound hemorrhoidal medical treatment using similar procedures and apparatus as for ultrasound uterine medical treatment.

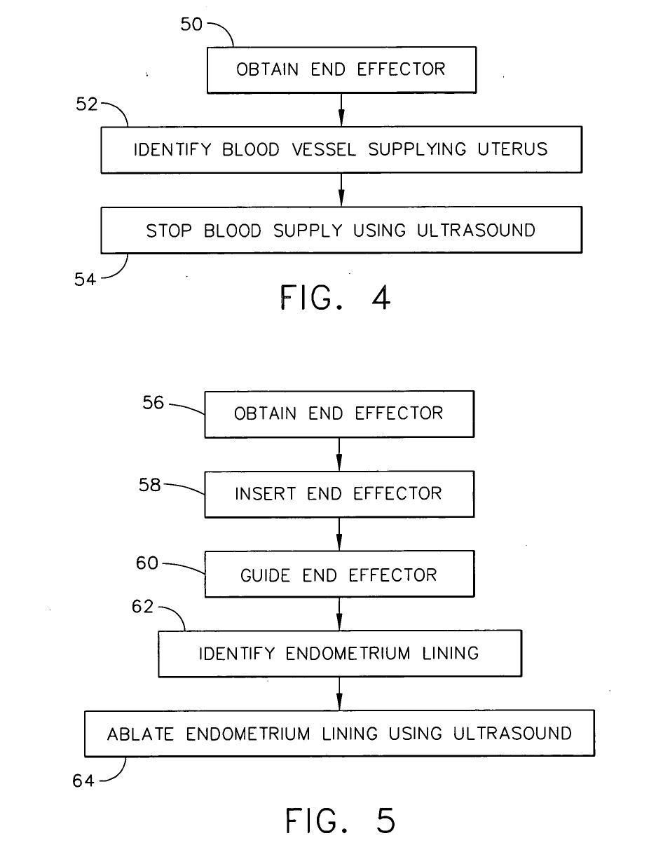

A second method of the invention is shown in block diagram form in FIG. 4 and is for ultrasound uterine medical treatment of a female patient. An embodiment of an ultrasound medical treatment system 10 which can be used to perform the second method is shown in FIG. 2. The second method includes steps a) through c). Step a) is labeled “Obtain End Effector” in block 50 of FIG. 4. Step a) includes obtaining an end effector 14 having an ultrasound medical-treatment transducer assembly 16. Step b) is labeled “Identify Blood Vessel Supplying Uterus” in block 52 of FIG. 4. Step b) includes identifying a blood vessel which supplies blood to a portion of the uterus of the patient. Step c) is labeled “Stop Blood Supply Using Ultrasound” in block 54 of FIG. 4. Step c) includes medically treating the blood vessel with ultrasound from the transducer assembly 16 to substantially seal the blood vessel to substantially stop the supply of blood from the blood vessel to the portion of the uterus.

In one implementation of the second method, not shown, the end effector 14 is an extracorporeal end effector. In one application of the second method, step b) identifies the blood vessel from ultrasound imaging using the transducer assembly 16. In one variation, the blood vessel of step d) includes blood containing an ultrasound contrast (echocontrast) agent for improved ultrasonic imaging, as can be appreciated by the artisan.

A third method of the invention is shown in block diagram form in FIG. 5 and is for ultrasound uterine medical treatment of a female patient. An embodiment of an ultrasound medical treatment system 10 which can be used to perform the third method is shown in FIG. 2. The third method includes steps a) through e). Step a) is labeled “Obtain End Effector” in block 56 of FIG. 5. Step a) includes obtaining an end effector 14 having an ultrasound medical-treatment transducer assembly 16. Step b) is labeled “Insert End Effector” in block 58 of FIG. 5. Step b) includes endoscopically inserting the end effector 14 into the patient. Step c) is labeled “Guide End Effector” in block 60 of FIG. 5. Step c) includes guiding the end effector within the patient into the uterine cavity. Step d) is labeled “Identify Endometrium Lining” in block 62 of FIG. 5. Step d) includes identifying the endometrium lining of the uterine cavity. Step e) is labeled “Ablate Endometrium Lining using Ultrasound” in block 64 of FIG. 5. Step e) includes medically treating the endometrium lining with ultrasound from the transducer assembly 16 to ablate a desired thickness (or even substantially the entire thickness) of at least a portion of (or even substantially the entire) endometrium lining to substantially stop abnormal uterine bleeding from the endometrium lining.

In the third method, the end effector 14 is a transvaginal end effector such as the endoscopic end effector 26 seen in FIG. 2. In one variation, step c) guides the endoscopic end effector 26 into the uterine cavity. In one modification, the endoscopic end effector 26 is an articulated end effector. In one construction, the endoscopic end effector 26 has a longitudinal axis 28, has at least two transversely-outwardly-facing ultrasound transducers 30 and 32 each having at least one ultrasound transducer element 34, and has a longitudinally-facing ultrasonic tip transducer 36. Other constructions are left to the artisan.

In one application of the third method, step d) identifies the endometrium lining of the uterine cavity from ultrasound imaging using the transducer assembly 16. In one variation, the endometrium lining of step d) includes blood containing an ultrasound contrast (echocontrast) agent for improved ultrasonic imaging, as can be appreciated by the artisan. In one extension of the second and/or third methods, there is also included the step of taking a biopsy of the uterus, wherein the end effector 14 also includes a biopsy tool (not shown).

In one enablement of any one or more of the first, second and third methods, as shown in FIG. 2, the ultrasound medical treatment system 10 also includes a handpiece 66 which is operatively connected to the end effector 14 and to an ultrasound controller 68, wherein the ultrasound controller 68 is operatively connected to a foot-pedal power switch 70, as can be appreciated by the artisan. In one variation, the handpiece 66 includes a control knob 72 used to articulate the end effector 14 and includes a control button 74 used to rotate the end effector 14, as is within the level of construction skill of the artisan.

Several benefits and advantages are obtained from one or more of the methods of the invention. In one example, ultrasound hemostasis (stoppage of blood flow) in a blood vessel supplying blood to a uterine fibroid is accomplished in a relatively non-invasive endoscopic or laparoscopic manner in a relatively short treatment time, because of the relatively small treated blood-vessel size and the relatively close distance between the ultrasound transducer and the blood vessel, which causes involution (shrinkage) of the uterine fibroid and stops any abnormal uterine bleeding from the uterine fibroid. In one extension, the uterine fibroid is also medically treated with ultrasound, in an endoscopic or laparoscopic manner, to ablate at least a part of the uterine fibroid. In another example, ultrasound ablation of at least a portion of the endometrium lining of the uterus is accomplished in a relatively non-invasive endoscopic manner and stops abnormal uterine bleeding from the endometrium lining of the uterus with excellent treatment depth control in ablating a desired thickness of endometrium lining because ultrasound can be focused to treat different depths within tissue.

While the present invention has been illustrated by a description of several methods and embodiments, it is not the intention of the applicants to restrict or limit the spirit and scope of the appended claims to such detail. Numerous other variations, changes, and substitutions will occur to those skilled in the art without departing from the scope of the invention. For instance, the ultrasound methods and systems of the invention have application in robotic assisted surgery taking into account the obvious modifications of such methods, systems and components to be compatible with such a robotic system. It will be understood that the foregoing description is provided by way of example, and that other modifications may occur to those skilled in the art without departing from the scope and spirit of the appended claims.

1. A method for ultrasound uterine medical treatment of a female patient comprising the steps of:
a) obtaining an end effector having an ultrasound medical-treatment transducer assembly;
b) inserting the end effector into the patient;
c) guiding the end effector within the patient to a region of patient tissue containing the uterus;
d) identifying a blood vessel in the region which supplies blood to a portion of the uterus; and
e) medically treating the blood vessel with ultrasound from the transducer assembly to substantially seal the blood vessel to substantially stop the supply of blood from the blood vessel to the portion of the uterus.

2. The method of claim 1, wherein step d) identifies the blood vessel from ultrasound imaging using the transducer assembly.

3. The method of claim 2, wherein the blood vessel includes blood containing an ultrasound contrast agent.

4. The method of claim 1, wherein the portion of the uterus includes a uterine fibroid, and wherein the blood vessel is a uterine artery, or a branch thereof, which supplies blood to the uterine fibroid.

5. The method of claim 4, also including the step of medically treating the uterine fibroid with ultrasound from the transducer assembly to ablate at least a part of the uterine fibroid.

6. The method of claim 5, also including the step of identifying the uterine fibroid at least in part from ultrasound imaging using the transducer assembly.

7. The method of claim 6, wherein the uterine fibroid includes blood containing an ultrasound contrast agent.

8. The method of claim 1, wherein the portion of the uterus includes a source of abnormal uterine bleeding, and wherein the blood vessel is a uterine artery, or a branch thereof, which supplies blood to the source of abnormal uterine bleeding.

9. The method of claim 8, wherein the source is a uterine fibroid.

10. The method of claim 1, wherein the end effector is an open-surgery end effector.

11. The method of claim 1, wherein the end effector is an endoscopic end effector.

12. The method of claim 11, wherein step c) guides the endoscopic end effector into the uterine cavity.

13. The method of claim 12, wherein the endoscopic end effector is an articulated end effector.

14. The method of claim 13, wherein the endoscopic end effector has a longitudinal axis, has at least two transversely-outwardly-facing ultrasound transducers each having at least one ultrasound transducer element, and has a longitudinally-facing ultrasonic tip transducer.

15. The method of claim 1, wherein the end effector is a laparoscopic end effector.

16. The method of claim 15, wherein the laparoscopic end effector comprises a tissue-retaining device including a first tissue-retaining member having an ultrasound medical-treatment transducer and including a second tissue-retaining member, and wherein step e) includes retaining the blood vessel between the first and second tissue-retaining members.

17. The method of claim 16, wherein the second tissue-retaining member includes an ultrasound reflector.

18. The method of claim 1, wherein the end effector is a catheter end effector, and wherein step c) guides the end effector inside the blood vessel.

19. The method of claim 1, wherein the end effector is a needle end effector.

20. A method for ultrasound uterine medical treatment of a female patient comprising the steps of:
a) obtaining an end effector having an ultrasound medical-treatment transducer assembly;
b) identifying a blood vessel which supplies blood to a portion of the uterus of the patient; and
c) medically treating the blood vessel with ultrasound from the transducer assembly to substantially seal the blood vessel to substantially stop the supply of blood from the blood vessel to the portion of the uterus.

21. The method of claim 20, wherein the end effector is an extracorporeal end effector.

22. The method of claim 21, wherein step b) identifies the blood vessel from ultrasound imaging using the transducer assembly.

23. The method of claim 22, wherein the blood vessel includes blood containing an ultrasound contrast agent.

24. A method for ultrasound uterine medical treatment of a female patient comprising the steps of:
a) obtaining an end effector having an ultrasound medical-treatment transducer assembly;
b) transvaginal-endoscopically inserting the end effector into the patient;
c) guiding the end effector within the patient into the uterine cavity;
d) identifying the endometrium lining of the uterine cavity; and
e) medically treating the endometrium lining with ultrasound from the transducer assembly to ablate a desired thickness of at least a portion of the endometrium lining to substantially stop abnormal uterine bleeding from the endometrium lining.

25. The method of claim 24, wherein the end effector is an articulated end effector.

26. The method of claim 25, wherein the end effector has a longitudinal axis, has at least two transversely-outwardly-facing ultrasound transducers each having at least one ultrasound transducer element, and has a longitudinally-facing ultrasonic tip transducer.

27. The method of claim 24, wherein step d) identifies the endometrium lining of the uterine cavity from ultrasound imaging using the transducer assembly.

28. The method of claim 27, wherein the endometrium lining includes blood containing an ultrasound contrast agent.
